(19) United States
(12) Patent Application Publication
(10) Pub. No.: US 2021/0208138 A1
(43) Pub. Date: Jul. 8, 2021

(54) COMPOSITE PARTICLE FOR IMMUNOCHROMATOGRAPHY, METHOD FOR MANUFACTURING THE SAME, AND IMMUNOCHROMATOGRAPHY

(71) Applicant: FUJIFILM Corporation, Tokyo (JP)

(72) Inventors: Yoshihiro ABURAYA, Ashigara-kami-gun (JP); Naoyuki NISHIKAWA, Ashigara-kami-gun (JP); Junichi KATADA, Ashigara-kami-gun (JP); Hiroyuki CHIKU, Ashigara-kami-gun (JP); Ayumi ERA, Ashigara-kami-gun (JP)

(73) Assignee: FUJIFILM Corporation, Tokyo (JP)

(21) Appl. No.: 17/205,649

(22) Filed: Mar. 18, 2021

Related U.S. Application Data
(63) Continuation of application No. PCT/JP2019/035874, filed on Sep. 12, 2019.

(30) Foreign Application Priority Data
Sep. 28, 2018 (JP) 2018-183910

(51) Int. Cl. G01N 33/543; C01G 49/08; G01N 33/531; G01N 33/553

(52) U.S. Cl. CPC G01N 33/54333; C01G 49/08; G01N 33/553; G01N 33/5434; G01N 33/531

(57) ABSTRACT

An object of the present invention is to provide a composite particle for an immunochromatography which has excellent spreadability, a method for manufacturing the same, and an immunochromatography using the composite particle for an immunochromatography. A composite particle for an immunochromatography of the present invention is a composite particle for an immunochromatography in which magnetic particles having an average particle size of 500 nm or less and gold particles having an average particle size of 500 nm or less are bound via an organic substance.

### CROSS-REFERENCE TO RELATED APPLICATIONS

This application is a Continuation of PCT International Application No. PCT/JP2019/035874 filed on Sep. 12, 2019, which claims priority under 35 U.S.C. § 119(a) to Japanese Patent Application No. 2018-183910 filed on Sep. 28, 2018. The above application is hereby expressly incorporated by reference, in its entirety, into the present application.

### BACKGROUND OF THE INVENTION

### 1. Field of the Invention

The present invention relates to a composite particle for an immunochromatography, a method for manufacturing the same, and an immunochromatography.

### 2. Description of the Related Art

An immunochromatography is a method generally used synonymously with immunochromatography. It is frequently used these days because operation is easy and measurement can be performed within a short period of time.

For example, in a case where an antigen such as influenza virus is detected by an immunochromatography, the following operation is performed.

First, a label modified with an antibody (labeled antibody) is prepared and mixed with a specimen containing an antigen. The labeled antibody binds to the antigen, and thereby a complex is formed. In a case where this complex is spread on an insoluble carrier having a detection line to which an antibody that specifically reacts with an antigen is applied, the complex reacts with the antibody on the detection line and is immobilized, and detection is confirmed visually or in other manners.

As a label used for such a labeled antibody, for example, JP2014-062920A discloses a composite particle obtained by binding of magnetic particles having a diameter of about 1 μm and gold particles having a diameter of about 50 nm via streptavidin-biotin (Example 1 and the like).

### SUMMARY OF THE INVENTION

Since it is necessary that a test substance such as an antigen be detected within a short period of time in an immunochromatography, a label to be used is required to be rapidly spread in an insoluble carrier by utilizing capillarity or the like. That is, a label used for an immunochromatography is required to exhibit excellent spreadability.

Under these circumstances, the inventors of the present invention have examined the composite particle formed of magnetic particles and gold particles disclosed in Examples of JP2014-062920A, and it became clear that spreadability of the composite particle does not satisfy a level required these days.

Accordingly, in view of the above circumstances, an object of the present invention is to provide a composite particle for an immunochromatography which has excellent spreadability, a method for manufacturing the same, and an immunochromatography using the composite particle for an immunochromatography.

As a result of intensive studies to achieve the above-mentioned object, the inventors of the present invention have found that the object can be achieved by specifying an average particle size of magnetic particles and gold particles, and therefore have completed the present invention.

That is, the inventors of the present invention have found that the object can be achieved by the following configurations.

(1) A composite particle for an immunochromatography, in which magnetic particles having an average particle size of 500 nm or less and gold particles having an average particle size of 500 nm or less are bound via an organic substance.

(2) The composite particle for an immunochromatography according to (1), in which an average particle size of at least one of the magnetic particles or the gold particles is less than 300 nm.

(3) The composite particle for an immunochromatography according to (2), in which an average particle size of the at least one of the magnetic particles or the gold particles is 150 nm or less.

(4) The composite particle for an immunochromatography according to (3), in which an average particle size of the at least one of the magnetic particles or the gold particles is less than 100 nm.

(5) The composite particle for an immunochromatography according to any one of (1) to (4), in which an average number of the gold particles bound to one of the magnetic particles via the organic substance is less than 10.0.

(6) The composite particle for an immunochromatography according to (5), in which an average number of the gold particles bound to one of the magnetic particles via the organic substance is 8.0 or less.

(7) The composite particle for an immunochromatography according to any one of (1) to (6), in which a surface of the magnetic particle is modified with a first organic substance, a surface of the gold particle is modified with a second organic substance, and the first organic substance and the second organic substance are bound via at least one selected from the group consisting of a chemical bond, an avidin-biotin interaction, a streptavidin-biotin interaction, a hydrophobic interaction, an electrostatic interaction, and an affinity interaction.

(8) A method for manufacturing a composite particle for an immunochromatography which is for manufacturing the composite particle for an immunochromatography according to any one of (1) to (7), the method comprising:

a preparation step of preparing modified magnetic particles that are magnetic particles modified with a first organic substance and having an average particle size of 500 nm or less, and modified gold particles that are gold particles modified with a second organic substance and having an average particle size of 500 nm or less; and

a mixing step of mixing the modified magnetic particles and the modified gold particles for binding of the first organic substance of the modified magnetic particles and the second organic substance of the modified gold particles via at least one selected from the group consisting of a chemical bond, an avidin-biotin interaction, a hydrophobic interaction, an electrostatic interaction, and an affinity interaction to obtain the composite particle for an immunochromatography.

(9) An immunochromatography using the composite particle for an immunochromatography according to any one of (1) to (7).

(10) The immunochromatography according to (9), comprising:

a mixing step of mixing a specimen that contains a test substance with a modified composite particle that is the composite particle for an immunochromatography according to any one of (1) to (7), which is modified with a first binding substance binding to the test substance, to obtain a complex of the test substance in the specimen and the modified composite particle;

a collection step of collecting, using magnetism, the complex in the specimen obtained after the mixing step;

a spreading step of spreading the complex collected in the collection step on an insoluble carrier having a reaction site at which a second binding substance binding to the test substance is immobilized; and

a trapping step of trapping the complex at the reaction site of the insoluble carrier.

(11) The immunochromatography according to (10), further comprising:

a silver amplification step of silver-amplifying the trapped complex subsequent to the trapping step.

As shown hereinafter, according to the present invention, it is possible to provide a composite particle for an immunochromatography which has excellent spreadability, a method for manufacturing the same, and an immunochromatography using the composite particle for an immunochromatography.

### DESCRIPTION OF THE PREFERRED EMBODIMENTS

Hereinafter, a composite particle for an immunochromatography of the embodiment of the present invention, a method of the embodiment of the present invention which is for manufacturing the composite particle for an immunochromatography, and an immunochromatography of the embodiment of the present invention which uses the composite particle for an immunochromatography will be described.

Numerical value ranges expressed using “to” in the present specification mean a range including numerical values described before and after “to” as a lower limit value and an upper limit value.

Furthermore, in the present specification, one kind of each component may be used alone, or two or more kinds thereof may be used in combination. In a case where two or more kinds of each component are used in combination, a content of the component indicates a total content unless otherwise specified.

[Composite Particle for Immunochromatography]

A composite particle for an immunochromatography of the embodiment of the present invention is a composite particle for an immunochromatography in which magnetic particles having an average particle size of 500 nm or less and gold particles having an average particle size of 500 nm or less are bound via an organic substance (hereinafter referred to as the “composite particle of the embodiment of the present invention”).

Hereinafter, each substance constituting the composite particle of the embodiment of the present invention will be described first, and then a method for manufacturing the composite particle of the embodiment of the present invention will be described.

[Magnetic Particle]

The magnetic particles constituting the composite particle of the embodiment of the present invention are not particularly limited as long as they are magnetic particles having an average particle size of 500 nm or less.

Since the composite particle of the embodiment of the present invention includes the magnetic particles, the composite particle can be easily collected using magnetism.

A material of the magnetic particles is not particularly limited as long as it is a material having magnetic properties, and specific examples thereof include iron, cobalt, nickel, oxides thereof, ferrite, alloys thereof, and the like. Among them, iron oxide is preferable because then spreadability, detection sensitivity, and collectability using magnetism are more excellent, and non-specific adsorption can be further suppressed. Hereinafter, the sentence, “spreadability, detection sensitivity, and collectability using magnetism are more excellent, and non-specific adsorption can be further suppressed” will also be referred to as the sentence, “the effects and the like of the present invention are more excellent.”

The magnetic particles may be particles obtained by molding only a material having magnetic properties into a particle form. Alternatively, the magnetic particles may be particles of which a surface has been coated with a polymer (such as polystyrene and silica gel) or the like and which has a material having magnetic properties as a core, or may be particles of which a surface has been coated using a material having magnetic properties and which has a polymer or the like as a core.

<Average Particle Size>

As described above, an average particle size of the magnetic particles is 500 nm or less. Among average particle sizes, an average particle size is preferably less than 400 nm, more preferably 300 nm or less, even more preferably less than 200 nm, and particularly preferably 150 nm or less for the reason that the effects and the like of the present invention are more excellent.

A lower limit of the average particle size of the magnetic particles is not particularly limited, but it is preferably 1 nm or more, more preferably 10 nm or more, and even more preferably 20 nm or more for the reason that collectability using magnetism is more excellent.

An average particle size of the magnetic particles is a value obtained as follows: at least 20 composite particles are observed with a transmission electron microscope (TEM), magnetic particles in the composite particles are specified by energy-dispersive X-ray spectroscopy (EDX), diameters of circles having the same area as a projected area of each of the magnetic particles are respectively calculated, and these diameters are arithmetically averaged.

[Gold Particles]

The gold particles constituting the composite particle of the embodiment of the present invention are not particularly limited as long as they are gold particles having an average particle size of 500 nm or less. The gold particles are preferably colloidal gold.

Since the composite particle of the embodiment of the present invention includes the gold particles, the composite particle is colored in a trapping step to be described later. That is, the composite particle can be used as a label for an immunochromatography. In addition, the composite particle also acts as a catalyst for reducing silver ions in a silver amplification step to be described later.

<Average Particle Size>

As described above, an average particle size of the gold particles is 500 nm or less. Among average particle sizes, an average particle size is preferably 300 nm or less, more preferably 200 nm or less, even more preferably 100 nm or less, and particularly preferably 50 nm or less for the reason that the effects and the like of the present invention are more excellent.

A lower limit of the average particle size of the gold particles is not particularly limited, but it is preferably 1 nm or more, more preferably 2 nm or more, and even more preferably 5 nm or more for the reason that detection sensitivity and silver amplification properties are more excellent.

An average particle size of the gold particles is a value obtained as follows: at least 20 composite particles are observed with a transmission electron microscope, gold particles in the composite particles are specified by EDX, diameters of circles having the same area as a projected area of each of the gold particles are respectively calculated, and these diameters are arithmetically averaged.

[Ratio of Average Particle Size of Magnetic Particles to Average Particle Size of Gold Particles]

A ratio of the average particle size of the gold particles to the average particle size of the magnetic particles (average particle size of gold particles/average particle size of magnetic particles) is not particularly limited, but it is preferably 0.01 to 10.0, more preferably 0.1 to 5.0, and even more preferably 0.2 to 3.0 for the reason that the effects and the like of the present invention are more excellent. Average particle size of gold particles/average particle size of magnetic particles is preferably less than 1.0 for the reason that the effects and the like of the present invention are more excellent.

[Organic Substance]

The organic substance constituting the composite particle of the embodiment of the present invention is not particularly limited, but it is preferably an avidin-biotin complex, a streptavidin-biotin complex, a condensate of an amine and a carboxylic acid, a reaction product of an amine and an epoxy compound (compound having an epoxy group), or a reaction product of a carboxylic acid and an epoxy compound, is more preferably an avidin-biotin complex or a streptavidin-biotin complex, and is even more preferably an avidin-biotin complex, for the reason that the effects and the like of the present invention are more excellent.

A mass of the organic substance is not particularly limited, but it is preferably 1,000 kDa or less for the reason that the effects and the like of the present invention are more excellent. A lower limit of the mass of the organic substance is not particularly limited, but it is 100 Da or more for the reason that the effects and the like of the present invention are more excellent.

[Number of Gold Particles Carried]

In the composite particle of the embodiment of the present invention, an average number of the gold particles bound to one of the magnetic particles via the organic substance (hereinafter, also referred to as “the number of the gold particles carried”) is not particularly limited, but it is preferably 100 or less, more preferably 50 or less, even more preferably 10 or less, particularly preferably 5.0 or less, and most preferably 3.0 or less, for the reason that the effects and the like of the present invention are more excellent. A lower limit of the number of the gold particles carried is not particularly limited, but it is 1.

The number of the gold particles carried is obtained as follows.

First, SEM-EDX analysis is performed on composite particles, a signal intensity derived from the magnetic particles and a signal intensity of Au are measured, and an amount ratio of the magnetic particles and the gold particles is obtained. Furthermore, the number of the gold particles carried is calculated by using the obtained amount ratio and the average particle size of the magnetic particles and the gold particles measured as described above.

[Average Particle Size of Composite Particles]

An average particle size of the composite particles of the embodiment of the present invention is not particularly limited, but it is preferably 10 to 600 nm, more preferably 20 to 300 nm, and even more preferably 50 to 300 nm, for the reason that the effects and the like of the present invention are more excellent.

An average particle size is a median diameter (d=50) measured by a dynamic light scattering method (measurement temperature: 25° C.).

[Suitable Aspect]

For the reason that the effects and the like of the present invention are more excellent, the composite particle of the embodiment of the present invention is preferably a composite particle for an immunochromatography in which a surface of the above-described magnetic particles is modified with a first organic substance, a surface of the above-described gold particles is modified with a second organic substance, and the first organic substance and the second organic substance are bound via at least one selected from the group consisting of a chemical bond, an avidin-biotin interaction, a hydrophobic interaction, an electrostatic interaction, and an affinity interaction.

A binding substance of the first organic substance and the second organic substance corresponds to the above-mentioned organic substance.

That is, the suitable aspect is a composite particle for an immunochromatography in which magnetic particles having an average particle size of 500 nm or less and gold particles having an average particle size of 500 nm or less are bound via the above-mentioned binding substance.

The first organic substance and the second organic substance are not particularly limited, but for the reason that the effects and the like of the present invention are more excellent, a combination of avidin and biotin, a combination of streptavidin and biotin, a combination of an amine and a carboxylic acid, a combination of an amine and an epoxy compound, and a combination of a carboxylic acid and an epoxy compound are preferable, a combination of avidin and biotin and a combination of streptavidin and biotin are more preferable, and a combination of avidin and biotin is even more preferable.

In the case of a combination of avidin and biotin, the first organic substance and the second organic substance are bound by an avidin-biotin interaction. In the case of a combination of streptavidin and biotin, the first organic substance and the second organic substance are bound by a streptavidin-biotin interaction. In the case of a combination of an amine and a carboxylic acid, the first organic substance and the second organic substance are bound by a chemical bond (amide bond). In the case of a combination of an amine and an epoxy compound, the first organic substance and the second organic substance are bound by a chemical bond. In the case of a combination of a carboxylic acid and an epoxy compound, the first organic substance and the second organic substance are bound by a chemical bond.

The above-described magnetic particles modified with the first organic substance are preferably magnetic particles coated with the first organic substance for the reason that the effects and the like of the present invention are more excellent.

Similarly, the above-described gold particles modified with the second organic substance are preferably gold particles coated with the second organic substance for the reason that the effects and the like of the present invention are more excellent.

[Manufacturing Method]

A method for manufacturing the above-described composite particle of the embodiment of the present invention is not particularly limited, but it is preferably a method including the following steps (1) and (2) (hereinafter, also referred to as “the manufacturing method of the embodiment of the present invention”), for the reason that the effects and the like of the present invention are more excellent in a composite particle to be obtained.

(1) Preparation Step

A step of preparing modified magnetic particles that are magnetic particles modified with a first organic substance and having an average particle size of 500 nm or less, and modified gold particles that are gold particles modified with a second organic substance and having an average particle size of 500 nm or less.

(2) Mixing Step

A step of mixing the modified magnetic particles and the modified gold particles for binding of the first organic substance of the modified magnetic particles and the second organic substance of the modified gold particles via at least one selected from the group consisting of a chemical bond, an avidin-biotin interaction, a hydrophobic interaction, an electrostatic interaction, and an affinity interaction to obtain the composite particle for an immunochromatography.

Hereinafter, the respective steps will be described.

<Preparation Step>

The preparation step is a step of preparing modified magnetic particles that are magnetic particles modified with a first organic substance and having an average particle size of 500 nm or less, and modified gold particles that are gold particles modified with a second organic substance and having an average particle size of 500 nm or less.

The magnetic particles, the gold particles, the first organic substance, and the second organic substance are as described above.

The modified magnetic particles and the modified gold particles are preferably prepared as a dispersion liquid from the viewpoint of ease of handling.

Commercially available products may be used for the modified magnetic particles (or a dispersion liquid thereof) and the modified gold particles (or a dispersion liquid thereof).

<Mixing Step>

The mixing step is a step of mixing the modified magnetic particles and the modified gold particles for binding of the first organic substance of the modified magnetic particles and the second organic substance of the modified gold particles via at least one selected from the group consisting of a chemical bond, an avidin-biotin interaction, a hydrophobic interaction, an electrostatic interaction, and an affinity interaction to obtain the composite particle for an immunochromatography.

It is preferable to incubate a mixture after mixing the modified magnetic particles and the modified gold particles from the viewpoint of sufficiently binding the first organic substance and the second organic substance.

In a case where the modified magnetic particles are a dispersion liquid, it is preferable to wash the particles, replace a buffer, and perform dilution using a phosphate buffer solution (PBS) or the like before the mixing step.

In a case where the modified gold particles are a dispersion liquid, it is preferable to wash the particles, replace a buffer, and perform dilution using a phosphate buffer solution (PBS) or the like before the mixing step.

In a case where the first organic substance and the second organic substance are a combination of an amine and a carboxylic acid, particles modified with carboxylic acid (magnetic particles or gold particles) are preferably caused to react with a dehydrating and condensing agent (for example, 1-ethyl-3-(3-dimethylaminopropyl)carbodiimide (EDC), N,N′-dicyclohexylcarbodiimide (DCC), and the like) before the mixing step. Accordingly, it is possible to efficiently cause these particles to react with particles modified with amine (magnetic particles or gold particles).

[Usage Applications]

As described above, since the composite particle of the embodiment of the present invention has excellent spreadability, it is useful for an immunochromatography. It is particularly useful for labeling in an immunochromatography (particularly immuno chromatography).

Since the composite particle of the embodiment of the present invention includes the magnetic particles, the composite particle can be easily collected using magnetism. Accordingly, a specimen in which a test substance is concentrated can be obtained, and thereby detection sensitivity of an immunochromatography can be significantly improved.

[Immunochromatography]

An immunochromatography of the embodiment of the present invention is an immunochromatography using the above-described composite particle of the embodiment of the present invention.

The immunochromatography of the embodiment of the present invention is preferably an immunochromatography including the following steps (1) to (4) (hereinafter, also referred to as “the chromatography of the embodiment of the present invention”).

(1) Mixing Step

A step of mixing a specimen that contains a test substance with a modified composite particle that is the above-described composite particle of the embodiment of the present invention, which is modified with a first binding substance that can bind to the test substance, to obtain a complex of the test substance in the specimen and the modified composite particle.

(2) Collection Step

A step of collecting, using magnetism, the complex in the specimen obtained after the mixing step.

(3) Spreading Step

A step of spreading the complex collected in the collection step on an insoluble carrier having a reaction site at which a second binding substance that can bind to the test substance is immobilized.

(4) Trapping Step

A step of trapping the complex at the reaction site of the insoluble carrier.

The chromatography of the embodiment of the present invention is an immunochromatography for detecting a test substance in a specimen.

Hereinafter, the respective steps will be described.

[Mixing Step]

The mixing step is a step of mixing a specimen that contains a test substance with a modified composite particle that is the above-described composite particle of the embodiment of the present invention, which is modified with a first binding substance that can bind to the test substance, to obtain a complex of the test substance in the specimen and the modified composite particle.

<Specimen>

The specimen used in the mixing step is not particularly limited as long as it is a specimen that can contain a test substance. Examples of such a specimen include biological specimens, particularly body fluids (for example, blood, serum, plasma, spinal fluid, tear fluid, sweat, urine, pus, runny nose, or sputum) or excrements (for example, feces), organs, tissues, mucous membranes and skin of animals (particularly humans); scraped test samples (swabs) and mouthwash that may contain these substances; or animals and plants themselves or dried substances thereof.

Examples of the test substances include natural substances, toxins, hormones, physiologically active substances such as pesticides, environmental pollutants, viruses, antigens, antibodies, and the like.

(Pretreatment of Specimen)

It is possible to use the above-mentioned specimen as it is, or in a form of an extraction liquid obtained by extracting the test specimen using an appropriate solvent for extraction, in a form of a diluent solution obtained by diluting an extraction liquid with an appropriate diluent, or in a form in which an extraction liquid has been concentrated by an appropriate method.

As the solvent for extraction, it is possible to use a solvent used in a general immunological analysis method (for example, water, physiological saline, a buffer solution, and the like), or a water-miscible organic solvent that enables performing of a direct antigen-antibody reaction by diluting with such a solvent.

<Modified Composite Particle>

The modified composite particle is the above-described composite particle of the embodiment of the present invention, which is modified with the first binding substance that can bind to the test substance. That is, the modified composite particle is the above-described composite particle of the embodiment of the present invention, and is a composite particle modified with the first binding substance that can bind to the test substance.

In the modified composite particle, a portion (site), which is to be modified with the first binding substance, of the above-described composite particle of the embodiment of the present invention is not particularly limited. For example, among the above-described composite particles of the embodiment of the present invention, the magnetic particles may be modified with the first binding substance, the gold particles may be modified with the first binding substance, or the organic substance may be modified with the first binding substance. Among them, it is preferable that the magnetic particles or the gold particles be modified with the first binding substance, and it is more preferable that the gold particles be modified with the first binding substance, for the reason that the effects and the like of the present invention are more excellent.

(First Binding Substance)

The first binding substance is not particularly limited as long as it is a compound having affinity for a test substance, and examples thereof include an antibody that specifically binds to a test substance consisting of an antigen; an antigen that specifically binds to a test substance consisting of an antibody; and an aptamer that binds to a test substance composed of a protein, a low-molecular weight compound, and the like.

In the chromatography of the embodiment of the present invention, it is preferable that the test substance be an antigen and the first binding substance be an antibody.

The above-mentioned antibody is not particularly limited. For example, it is possible to use antisera prepared from animal sera immunized with the test substance, immunoglobulin fractions purified from antisera, monoclonal antibodies obtained by cell fusion using animal spleen cells immunized with the test substance, or fragments thereof [for example, F(ab′)₂, Fab, Fab′, or Fv]. Preparation of these antibodies can be performed by a conventional method.

<Method for Manufacturing Modified Composite Particle>

A method for manufacturing the modified composite particle is not particularly limited, and examples thereof include the following methods (1) and (2). Among them, the following method (1) is preferable for the reason that the effects and the like of the present invention are more excellent with respect to modified composite particles to be obtained.

Method (1) in which magnetic particles or gold particles modified with the first binding substance are used as magnetic particles or gold particles in the above-described manufacturing method of the embodiment of the present invention.

Method (2) in which the above-mentioned composite particle of the embodiment of the present invention is modified with the first binding substance.

In (1), examples of methods of modifying gold particles with the first binding substance include a conventionally known method described below (for example, The Journal of Histochemistry and Cytochemistry, 30, 7 (1982) 691-696). As a specific example, gold particles and the first binding substance (for example, an antibody) are mixed in an appropriate buffer solution at room temperature condition for 5 minutes or longer. After the reaction, a precipitate obtained by centrifugation is dispersed in a solution containing a dispersant such as polyethylene glycol, and thereby gold particles modified with the first binding substance can be obtained.

<Mixing of Specimen and Modified Composite Particle>

In the mixing step, the specimen and the modified composite particle are mixed.

Accordingly, in a case where the specimen contains a test substance, the test substance in the specimen reacts with the first binding substance of the modified composite particle, and thereby a complex of the test substance and the modified composite particle is formed in the specimen. On the other hand, in a case where the specimen does not contain a test substance, a complex is not formed.

[Collection Step]

The collection step is a step of collecting, using magnetism, the complex in the specimen obtained after the mixing step.

In a case where the composite particles of the embodiment of the present invention (unreacted composite particles) which did not react with the test substance are present in the specimen obtained after the mixing step, the unreacted composite particles are also collected together. In addition, in a case where the specimen does not contain a test substance, a complex is not formed in the mixing step, and the unreacted composite particles are collected.

In the collection step, by collecting the complex and/or the unreacted composite particles (hereinafter, also collectively referred to as the “complex and the like”), it is possible to perform, for example, operations such as increasing a concentration of the complex and the like in the specimen, and separating the complex and the like. Accordingly, detection sensitivity and a signal noise ratio (SN ratio) can be significantly improved.

A method of collecting, using magnetism, the complex and the like in the specimen obtained after the mixing step is not particularly limited, and examples thereof include a method of putting the specimen obtained after the mixing step into a microtube installed on a magnetic stand, and the like.

[Spreading Step]

A spreading step is a step of spreading the complex collected in the collection step on an insoluble carrier having a reaction site at which a second binding substance binding to the test substance is immobilized.

<Insoluble Carrier>

The above-mentioned insoluble carrier is an insoluble carrier having a reaction site (test line) at which a second binding substance that can bind to the test substance is immobilized.

(Insoluble Carrier)

The insoluble carrier is preferably a porous carrier. A nitrocellulose membrane, a cellulose membrane, an acetyl cellulose membrane, a polysulfone membrane, a polyether sulfone membrane, a nylon membrane, a glass fiber, a non-woven fabric, a cloth, a thread, and the like are particularly preferable.

(Second Binding Substance)

Specific examples and a suitable aspect of the second binding substance are the same as those of the above-described first binding substance. The second binding substance may be the same as or different from the above-described first binding substance.

<Spreading of Complex>

In the spreading step, the complex is spread on the insoluble carrier. For example, a specimen containing the complex is spread in a horizontal direction of the insoluble carrier by flowing the specimen containing the complex concentrated in the collection step from one end of the insoluble carrier.

As described above, in the case where the composite particles of the embodiment of the present invention (unreacted composite particles) which did not react with the test substance are present in the specimen obtained after the mixing step, since the unreacted composite particles are also collected together in the collection step, the unreacted composite particles are also spread together with the complex. In addition, in the case where the specimen does not contain a test substance, the complex is not formed in the mixing step and only the unreacted composite particles are collected in the collection step, and therefore the unreacted composite particles are spread.

[Trapping Step]

A trapping step is a step of trapping the complex at the reaction site of the insoluble carrier.

As described above, since the second binding substance capable of binding to a test substance is immobilized at the reaction site of the insoluble carrier, the complex spread on the insoluble carrier in the spreading step (complex of a test substance and a modified composite particle) is trapped at the reaction site (test line) of the insoluble carrier.

The trapped complex is visible because it is colored by surface plasmon of gold particles and the like. In addition, it is also possible to estimate a concentration of the trapped complex using an image analysis device or the like. In this manner, the test substance in the specimen can be detected.

In a case where a specimen does not contain a test substance, the complex is not formed, and therefore the complex is not trapped at the reaction site of the insoluble carrier and is not colored.

[Silver Amplification Step]

It is preferable that the chromatography of the embodiment of the present invention further include a silver amplification step after the above-described trapping step.

The silver amplification step is a step of forming large silver particles in the complex trapped at the reaction site of the insoluble carrier by providing silver ions to the insoluble carrier after the trapping step. More specifically, it is a step in which silver ions are reduced using gold particles of the complex as a catalyst to form silver particles (for example, a diameter of 10 μm or more).

Accordingly, visibility and a signal noise ratio (SN ratio) of the trapped complex are remarkably improved.

<Suitable Aspect>

A method of providing silver ions to the insoluble carrier after the trapping step is not particularly limited, but a method in which the following reducing agent solution and the following amplification liquid are used is preferable because then visibility and an SN ratio are further improved.

(Reducing agent solution)

The reducing agent solution contains a reducing agent capable of reducing silver ions. As the reducing agent capable of reducing silver ions, any inorganic or organic material or a mixture thereof can be used as long as it can reduce silver ions to silver. Preferred examples of inorganic reducing agents include reducing metal salts and reducing metal complex salts which are capable of changing an atomic value with metal ions such as Fe²⁺, V²⁺, or Ti³⁺. In a case in which an inorganic reducing agent is used, it is necessary to remove or detoxify oxidized ions by complexing or reducing the oxidized ions. For example, in a system in which Fe²⁺ is used as the reducing agent, a complex of Fe³⁺, which is an oxide, is formed using citric acid or ethylenediaminetetraacetic acid (EDTA), and therefore detoxification is possible. In the present invention, it is preferable to use such an inorganic reducing agent, and as a more preferable aspect of the present invention, it is preferable to use a metal salt of Fe²⁺ as the reducing agent.

It is also possible to use a main developing agent used in a light-sensitive silver halide photographic material of a wet-type (such as methyl gallate, hydroquinone, substituted hydroquinone, 3-pyrazolidones, p-aminophenols, p-phenylenediamines, hindered phenols, amidoximes, azines, catechols, pyrogallols, ascorbic acid (or derivatives thereof), and leuco colorants), and other materials obvious to those who are skilled in the technology in the present field, such as a material disclosed in U.S. Pat. No. 6,020,117A.

As the reducing agent, an ascorbic acid reducing agent is also preferable. Useful ascorbic acid reducing agents include ascorbic acid and analogs thereof, and isomers and derivatives thereof. Preferred examples thereof include D- or L-ascorbic acid and sugar derivatives thereof (such as γ-lactoascorbic acid, glucoascorbic acid, fucoascorbic acid, glucoheptoascorbic acid, and maltoascorbic acid), a sodium salt of ascorbic acid, a potassium salt of ascorbic acid, isoascorbic acid (or L-erythroascorbic acid), salts thereof (such as alkali metal salts, ammonium salts, or salts known in the technical field), ascorbic acid of the enediol type, ascorbic acid of the enaminol type, ascorbic acid of the thioenol type, and the like. Particularly preferred examples thereof include D-, L-, or D,L-ascorbic acid (and an alkali metal salt thereof) or isoascorbic acid (or an alkali metal salt thereof), and a sodium salt is a preferable salt. A mixture of these reducing agents can be used as necessary.

(Amplification Liquid)

The amplification liquid is a liquid containing a compound containing silver ions. As the compound containing silver ions, it is possible to use, for example, organic silver salts, inorganic silver salts, or silver complexes. The compound is preferably a silver ion-containing compound having a high solubility in solvents such as water, and examples thereof include silver nitrate, silver acetate, silver lactate, silver butyrate, silver thiosulfate, and the like. Silver nitrate is particularly preferred. Silver complexes are preferably silver complexes coordinated with ligands having a water-soluble group such as a hydroxyl group or a sulfone group, and examples thereof include silver hydroxythioether and the like.

The organic silver salts, the inorganic silver salts, or the silver complexes are contained in the amplification liquid as silver at a concentration of 0.001 mol/L to 5 mol/L, preferably 0.005 mol/L to 3 mol/L, and more preferably 0.01 mol/L to 1 mol/L.

Examples of auxiliaries of the amplification liquid include buffers, preservatives such as antioxidants or organic stabilizers, rate regulators, and the like. As the buffer, it is possible to use, for example, a buffer formed of acetic acid, citric acid, sodium hydroxide, or salts of any of these substances, or tris(hydroxymethyl)aminomethane or other buffers used in general chemical experiments. A pH can be adjusted to an optimum pH for the amplification liquid by appropriately using these buffers. Furthermore, as an antifogging agent, an alkylamine can be used as an auxiliary, and dodecylamine is particularly preferable. In addition, a surfactant can be used in order to improve solubility of these auxiliaries, and C₉H₁₉—C₆H₄—O—(CH₂CH₂O)₅₀H is particularly preferable.

### EXAMPLES

Hereinafter, the present invention will be described in more detail with reference to examples, but the present invention is not limited thereto.

[Manufacturing of Particles]

Particles were manufactured as follows.

Composite particles of Examples 1 to 17 are composite particles obtained by binding of gold particles having an average particle size of 500 nm or less and magnetic particles having an average particle size of 500 nm or less via an organic substance, and therefore the composite particles correspond to the composite particles of the embodiment of the present invention.

On the other hand, particles of Comparative Examples 1 and 2 are particles formed of only magnetic particles, and therefore these particles do not correspond to the composite particles of the embodiment of the present invention.

Furthermore, composite particles of Comparative Example 3 are composite particles obtained by binding of gold particles and magnetic particles via an organic substance, but because an average particle size of the magnetic particles is more than 500 nm, these composite particles do not correspond to the composite particles of the embodiment of the present invention.

### Example 1

The composite particles of Example 1 were manufactured as follows.

(Preparation Step)

First, as follows, magnetic particles modified with avidin (modified magnetic particles) and gold particles modified with biotin (modified gold particles) were prepared.

(1) Magnetic Particles Modified with Avidin

A dispersion liquid of magnetic particles (iron oxide) having an average particle size of 30 nm (manufactured by Sigma-Aldrich Co. LLC) and avidin were mixed and stirred at 27° C. for 1 hour, and thereby a dispersion liquid of magnetic particles modified with avidin (first organic substance) was obtained.

(2) Gold Particles Modified with Biotin

A dispersion liquid of gold particles (gold colloid) having an average particle size of 50 nm (manufactured by Cosmo Bio Co., Ltd.) and biotin were mixed and stirred at 27° C. for 1 hour, and thereby a dispersion liquid of gold particles modified with biotin (second organic substance) was obtained.

(Mixing Step)

Washing of particles, buffer replacement, and dilution were performed using phosphate buffered saline (PBS) for the obtained dispersion liquid of magnetic particles modified with avidin. In addition, washing of particles, buffer replacement, and dilution were performed using phosphate buffered saline (PBS) for the obtained dispersion liquid of gold particles modified with biotin. Thereafter, the two dispersion liquids were mixed and incubated for 30 minutes.

As a result, composite particles (composite particles obtained by binding of magnetic particles (average particle size: 30 nm) and gold particles (average particle size: 50 nm) via an avidin-biotin complex), which are to be obtained by binding of avidin of magnetic particles (average particle size: 30 nm) modified with avidin and biotin of gold particles (average particle size: 50 nm) modified with biotin by an avidin-biotin interaction, were obtained.

A microtube was installed on a magnetic stand, and B/F separation was performed on the obtained dispersion liquid in order to separate unreacted gold particles from the obtained dispersion liquid of composite particles.

### Example 2

The composite particles of Example 2 were manufactured as follows.

(Preparation Step)

First, as follows, magnetic particles modified with amine (modified magnetic particles) and gold particles modified with carboxylic acid (modified gold particles) were prepared.

(1) Magnetic Particles Modified with Amine

A dispersion liquid of magnetic particles (iron oxide) having an average particle size of 30 nm (manufactured by Sigma-Aldrich Co. LLC) and amine were mixed and stirred at 27° C. for 1 hour, and thereby a dispersion liquid of magnetic particles modified with amine (first organic substance) was obtained.

(2) Gold Particles Modified with Carboxylic Acid

A dispersion liquid of gold particles (gold colloid) having an average particle size of 50 nm (manufactured by Cosmo Bio Co., Ltd.) and carboxylic acid were mixed and stirred at 27° C. for 1 hour, and thereby a dispersion liquid of gold particles modified with carboxylic acid (second organic substance) was obtained.

(Mixing Step)

Washing of particles, buffer replacement, and dilution were performed using phosphate buffered saline (PBS) for the obtained dispersion liquid of magnetic particles modified with amine. In addition, washing of particles, buffer replacement, and dilution were performed using phosphate buffered saline (PBS) for the obtained dispersion liquid of gold particles modified with carboxylic acid. 1-Ethyl-3-(3-dimethylaminopropyl)carbodiimide (EDC) was added into the dispersion liquid of gold particles modified with carboxylic acid, and the mixture was slowly stirred for about 10 minutes. Thereafter, the two dispersion liquids were mixed and incubated for 30 minutes.

As a result, composite particles (composite particles obtained by binding of magnetic particles (average particle size: 30 nm) and gold particles (average particle size: 50 nm) via a condensate of amine and carboxylic acid), which are to be obtained by binding of amine of magnetic particles (average particle size: 30 nm) modified with amine and carboxylic acid of gold particles (average particle size: 50 nm) modified with carboxylic acid by a chemical bond, were obtained.

A microtube was installed on a magnetic stand, and B/F separation was performed three times on the obtained dispersion liquid in order to separate unreacted gold particles from the obtained dispersion liquid of composite particles.

### Example 3

The composite particles of Example 3 were manufactured as follows.

(Preparation Step)

First, as follows, magnetic particles modified with carboxylic acid (modified magnetic particles) and gold particles modified with amine (modified gold particles) were prepared.

(1) Magnetic Particles Modified with Carboxylic Acid

A dispersion liquid of magnetic particles (iron oxide) having an average particle size of 30 nm (manufactured by Sigma-Aldrich Co. LLC) and carboxylic acid were mixed and stirred at 27° C. for 1 hour, and thereby a dispersion liquid of magnetic particles modified with carboxylic acid (first organic substance) was obtained.

(2) Gold Particles Modified with Amine

A dispersion liquid of gold particles (gold colloid) having an average particle size of 50 nm (manufactured by Cosmo Bio Co., Ltd.) and amine were mixed and stirred at 27° C. for 1 hour, and thereby a dispersion liquid of gold particles modified with amine (second organic substance) was prepared.

(Mixing Step)

Washing of particles, buffer replacement, and dilution were performed using phosphate buffered saline (PBS) for the obtained dispersion liquid of magnetic particles modified with carboxylic acid. In addition, washing of particles, buffer replacement, and dilution were performed using phosphate buffered saline (PBS) for the obtained dispersion liquid of gold particles modified with amine. 1-Ethyl-3-(3-dimethylaminopropyl)carbodiimide (EDC) was added into the dispersion liquid of magnetic particles modified with carboxylic acid, and the mixture was slowly stirred for about 10 minutes. Thereafter, the two dispersion liquids were mixed and incubated for 30 minutes.

As a result, composite particles (composite particles obtained by binding of magnetic particles (average particle size: 30 nm) and gold particles (average particle size: 50 nm) via a condensate of carboxylic acid and amine), which are to be obtained by binding of carboxylic acid of magnetic particles (average particle size: 30 nm) modified with carboxylic acid and amine of gold particles (average particle size: 50 nm) modified with amine by a chemical bond, were obtained.

A microtube was installed on a magnetic stand, and B/F separation was performed three times on the obtained dispersion liquid in order to separate unreacted gold particles from the obtained dispersion liquid of composite particles.

### Examples 4 to 8

A dispersion liquid of each of the composite particles was obtained according to the same procedure as in Example 1 except that a dispersion liquid of magnetic particles (iron oxide) shown in Table 1 was used as a dispersion liquid of magnetic particles used to prepare the dispersion liquid of magnetic particles modified with avidin, and a dispersion liquid of gold particles shown in Table 1 was used as a dispersion liquid of gold particles used to prepare the dispersion liquid of gold particles modified with biotin.

### Examples 9 to 17 and Comparative Example 3

A dispersion liquid of each of the composite particles was obtained according to the same procedure as in Example 3 except that a dispersion liquid of magnetic particles (iron oxide) shown in Table 1 was used as a dispersion liquid of magnetic particles used to prepare the dispersion liquid of magnetic particles modified with carboxylic acid, and a dispersion liquid of gold particles shown in Table 1 was used as a dispersion liquid of gold particles used to prepare the dispersion liquid of gold particles modified with amine. In Example 15, a proportion of a dispersion liquid of gold particles was increased in a case where two dispersion liquids were mixed.

### Comparative Example 1

A dispersion liquid of magnetic particles (iron oxide) having an average particle size of 30 nm (manufactured by Sigma-Aldrich Co. LLC) and avidin were mixed and stirred at 27° C. for 1 hour, and thereby a dispersion liquid of magnetic particles modified with avidin was obtained. The obtained magnetic particles were used for Comparative Example 1.

### Comparative Example 2

A dispersion liquid of magnetic particles (iron oxide) having an average particle size of 1,000 nm (manufactured by Nanocs Inc.) and carboxylic acid were mixed and stirred at 27° C. for 1 hour, and thereby a dispersion liquid of magnetic particles modified with carboxylic acid was obtained. The obtained magnetic particles were used for Comparative Example 2.

[Evaluation]

The following evaluation was performed on the obtained particles.

<Number of Gold Particles Carried>

The number of the gold particles carried was obtained for the obtained composite particles (Examples 1 to 17 and Comparative Example 3).

Specifically, the obtained dispersion liquid of the composite particles was added dropwise onto a glass substrate, dried, and subjected to SEM-EDX analysis. Next, an amount ratio of the magnetic particles and the gold particles was obtained from a signal intensity of Fe and a signal intensity of Au. Furthermore, the number of the gold particles carried was obtained using the obtained amount ratio, and the average particle size of the magnetic particles and the gold particles.

Evaluation results are shown in Table 1.

<Spreadability>

Spreadability of the obtained particles was evaluated as follows.

A membrane (insoluble carrier) (porous carrier) used for an influenza inspection kit was removed. Thereafter, the dispersion liquid of the particles of the examples and the comparative examples, in which a concentration of the particles was adjusted to 10⁶ particles/ml, was used for a test liquid (a), and 200 μL of the test liquid (a) was flowed from one end of the membrane. After 300 seconds had passed, particles that reached the other end of the membrane were recovered separately, 200 μL of a dispersion buffer solution was added to obtain a test liquid (b). Each of the liquids was added dropwise onto a glass substrate in the same amount and at the same dilution ratio and dried. An average number of the particles was calculated from a scanning electron microscope (SEM) image of this glass substrate, and ratios of the particles were analyzed. The average number of the particles referred to herein is an average value of the number of particles in 10 randomly captured SEM images.

Next, spreadability was evaluated as follows: a case in which a ratio (passage probability) of the number of recovered particles to the number of flowed particles was equal to or more than 40%: “A,” a case in which a ratio thereof was equal to or more than 30% and less than 40%: “B,” a case in which a ratio thereof was equal to or more than 20% and less than 30%: “C,” and a case in which a ratio thereof was less than 20%: “D.”

Evaluation results are shown in Table 1. Practically, A, B, or C is preferable, A or B is more preferable, and A is even more preferable from the viewpoint of spreadability.

<Silver Amplification Properties>

Silver amplification properties of the obtained particles were evaluated as follows.

(Production of Reducing Agent Solution)

23.6 mL of an aqueous solution of 1 mol/L iron nitrate, which was produced by dissolving iron(III) nitrate nonahydrate (product number 095-00995, manufactured by FUJIFILM Wako Pure Chemical Corporation) in water, and 13.1 g of citric acid (product number 038-06925, manufactured by FUJIFILM Wako Pure Chemical Corporation) were dissolved in 290 g of water. After all of the substances were dissolved, 36 mL of nitric acid (10 wt %) was added thereto while stirring with a stirrer, 60.8 g of ammonium iron(II) sulfate hexahydrate (product number 091-00855, manufactured by FUJIFILM Wako Pure Chemical Corporation) was further added, and the resultant solution was used for a reducing agent solution.

(Production of Amplification Liquid)

8 mL of a silver nitrate solution (including 10 g of silver nitrate) and 24 mL of an aqueous solution of 1 mol/L iron nitrate were added to 66 g of water. Furthermore, this solution was mixed with a solution obtained by dissolving 5.9 mL of nitric acid (10 wt %), 0.1 g of dodecylamine (product number 123-00246, manufactured by FUJIFILM Wako Pure Chemical Corporation), and 0.1 g of a surfactant C₁₂H₂₅—C₆H₄—O—(CH₂CH₂O)₅₀H in 47.6 g of water in advance, and the resultant solution was used for an amplification liquid.

(Silver Amplification)

The obtained particles were added dropwise onto a glass substrate and dried. Thereafter, the reducing agent solution and the amplification liquid produced as described above were added dropwise on the dried particles in this order and dried. Next, SEM-EDX analysis was performed, and silver amplification properties were evaluated as follows: a case in which a signal intensity of Ag was equal to or more than a signal intensity of Fe: “A,” and a case in which a signal intensity of Ag was equal to or less than half a signal intensity of Fe: “D.”

Evaluation results are shown in Table 1. Practically, A is preferable from the viewpoint of silver amplification properties.

TABLE 1

| | Magnetic particles | | | | Gold particles | | | | | |
|---|---|---|---|---|---|---|---|---|---|---|
| | Dispersion liquid of | | | | Dispersion liquid of | | | Evaluation | | |
| | magnetic particles | | | | gold particles | | | Number | | |
| | Average | | | | Average | | | of gold | | Silver |
| | particle | | | First organic | particle | | Second organic | particles | | amplification |
| Table 1 | size [nm] | | Manufacturer | substance | size [nm] | Manufacturer | substance | carried | Spreadability | properties |
| Example 1 | 30 | | Sigma-Aldrich | Avidin | 50 | Cosmo Bio | Biotin | 1.4 | A | A |
| | | | Co. LLC | | | | | | | |
| Example 2 | 30 | | Sigma-Aldrich | Amine | 50 | Cosmo Bio | Carboxylic acid | 1.1 | A | A |
| | | | Co. LLC | | | | | | | |
| Example 3 | 30 | | Sigma-Aldrich | Carboxylic acid | 50 | Cosmo Bio | Amine | 1.2 | A | A |
| | | | Co. LLC | | | | | | | |
| Example 4 | 30 | | Sigma-Aldrich | Avidin | 40 | Cosmo Bio | Biotin | 1.4 | A | A |
| | | | Co. LLC | | | | | | | |
| Example 5 | 30 | | Sigma-Aldrich | Avidin | 30 | Cosmo Bio | Biotin | 1.5 | A | A |
| | | | Co. LLC | | | | | | | |
| Example 6 | 30 | | Sigma-Aldrich | Avidin | 20 | Cosmo Bio | Biotin | 1.6 | A | A |
| | | | Co. LLC | | | | | | | |
| Example 7 | 25 | to 30 | Nanocs | Avidin | 40 | Cosmo Bio | Biotin | 1.3 | A | A |
| Example 8 | 20 | | Sigma-Aldrich | Avidin | 40 | Cosmo Bio | Biotin | 1.1 | A | A |
| | | | Co. LLC | | | | | | | |
| Example 9 | 160 | | NANOBRICK | Carboxylic acid | 50 | Cosmo Bio | Amine | 4.2 | A | A |
| Example 10 | 160 | | NANOBRICK | Carboxylic acid | 30 | Cosmo Bio | Amine | 8.6 | B | A |
| Example 11 | 100 | to 200 | Nanocs | Carboxylic acid | 50 | Cosmo Bio | Amine | 4.4 | A | A |
| Example 12 | 100 | to 200 | Nanocs | Carboxylic acid | 30 | Cosmo Bio | Amine | 8.9 | B | A |
| Example 13 | 160 | to 240 | COREFRONT | Carboxylic acid | 50 | Cosmo Bio | Amine | 5.1 | A | A |
| Example 14 | 160 | to 240 | COREFRONT | Carboxylic acid | 30 | Cosmo Bio | Amine | 9.6 | B | A |
| Example 15 | 160 | to 240 | COREFRONT | Carboxylic acid | 50 | Cosmo Bio | Amine | 20 | B | A |
| Example 16 | 300 | to 500 | COREFRONT | Carboxylic acid | 50 | Cosmo Bio | Amine | 7.4 | C | A |
| Example 17 | 300 | to 500 | COREFRONT | Carboxylic acid | 10 | Cosmo Bio | Amine | 4.6 | C | A |
| Comparative | 30 | | Sigma-Aldrich | Avidin | Not used | | | 0 | A | D |
| Example 1 | | | Co. LLC | | | | | | | |
| Comparative | 1000 | | Nanocs | Carboxylic acid | Not used | | | 0 | D | D |
| Example 2 | | | | | | | | | | |
| Comparative | 1000 | | Nanocs | Carboxylic acid | 50 | Cosmo Bio | Amine | 50 | D | A |
| Example 3 | | | | | | | | | | |

As can be seen from Table 1, in all of Examples 1 to 17 which were the composite particles in which an average particle size of the magnetic particles was 500 nm or less, excellent spreadability (passage probability of 20% or more) (level that can withstand practical use) was exhibited as compared with Comparative Example 3 which was the composite particles in which an average particle size of the magnetic particles was more than 500 nm. Among them, in Examples 1 to 15 in which an average particle size of the magnetic particles was less than 300 nm, more excellent spreadability (passage probability of 30% or more) (level that enables improvement of measurement accuracy and realization of high-sensitivity) was exhibited. Among them, in Examples 1 to 9, 11, and 13 in which the number of the gold particles carried was 8.0 or less, further excellent spreadability (passage probability of 40% or more) was exhibited.

1. A composite particle for an immunochromatography, in which magnetic particles having an average particle size of 500 nm or less and gold particles having an average particle size of 500 nm or less are bound via an organic substance.

2. The composite particle for an immunochromatography according to claim 1,
wherein an average particle size of at least one of the magnetic particles or the gold particles is less than 300 nm.

3. The composite particle for an immunochromatography according to claim 2,
wherein an average particle size of the at least one of the magnetic particles or the gold particles is 150 nm or less.

4. The composite particle for an immunochromatography according to claim 3,
wherein an average particle size of the at least one of the magnetic particles or the gold particles is less than 100 nm.

5. The composite particle for an immunochromatography according to claim 1,
wherein an average number of the gold particles bound to one of the magnetic particles via the organic substance is less than 10.0.

6. The composite particle for an immunochromatography according to claim 5,
wherein an average number of the gold particles bound to one of the magnetic particles via the organic substance is 8.0 or less.

7. The composite particle for an immunochromatography according to claim 1,
wherein a surface of the magnetic particle is modified with a first organic substance,
a surface of the gold particle is modified with a second organic substance, and
the first organic substance and the second organic substance are bound via at least one selected from the group consisting of a chemical bond, an avidin-biotin interaction, a streptavidin-biotin interaction, a hydrophobic interaction, an electrostatic interaction, and an affinity interaction.

8. A method for manufacturing a composite particle for an immunochromatography which is for manufacturing the composite particle for an immunochromatography according to claim 1, the method comprising:
a preparation step of preparing modified magnetic particles that are magnetic particles modified with a first organic substance and having an average particle size of 500 nm or less, and modified gold particles that are gold particles modified with a second organic substance and having an average particle size of 500 nm or less; and
a mixing step of mixing the modified magnetic particles and the modified gold particles for binding of the first organic substance of the modified magnetic particles and the second organic substance of the modified gold particles via at least one selected from the group consisting of a chemical bond, an avidin-biotin interaction, a hydrophobic interaction, an electrostatic interaction, and an affinity interaction to obtain the composite particle for an immunochromatography.

9. An immunochromatography using the composite particle for an immunochromatography according to claim 1.

10. The immunochromatography according to claim 9, comprising:
a mixing step of mixing a specimen that contains a test substance with a modified composite particle that is the composite particle for an immunochromatography, which is modified with a first binding substance binding to the test substance, to obtain a complex of the test substance in the specimen and the modified composite particle;
a collection step of collecting, using magnetism, the complex in the specimen obtained after the mixing step;
a spreading step of spreading the complex collected in the collection step on an insoluble carrier having a reaction site at which a second binding substance binding to the test substance is immobilized; and
a trapping step of trapping the complex at the reaction site of the insoluble carrier.

11. The immunochromatography according to claim 10, further comprising:
a silver amplification step of silver-amplifying the trapped complex subsequent to the trapping step.

12. The composite particle for an immunochromatography according to claim 2,
wherein an average number of the gold particles bound to one of the magnetic particles via the organic substance is less than 10.0.

13. The composite particle for an immunochromatography according to claim 12,
wherein an average number of the gold particles bound to one of the magnetic particles via the organic substance is 8.0 or less.

14. The composite particle for an immunochromatography according to claim 2,
wherein a surface of the magnetic particle is modified with a first organic substance,
a surface of the gold particle is modified with a second organic substance, and
the first organic substance and the second organic substance are bound via at least one selected from the group consisting of a chemical bond, an avidin-biotin interaction, a streptavidin-biotin interaction, a hydrophobic interaction, an electrostatic interaction, and an affinity interaction.

15. A method for manufacturing a composite particle for an immunochromatography which is for manufacturing the composite particle for an immunochromatography according to claim 2, the method comprising:
a preparation step of preparing modified magnetic particles that are magnetic particles modified with a first organic substance and having an average particle size of 500 nm or less, and modified gold particles that are gold particles modified with a second organic substance and having an average particle size of 500 nm or less; and
a mixing step of mixing the modified magnetic particles and the modified gold particles for binding of the first organic substance of the modified magnetic particles and the second organic substance of the modified gold particles via at least one selected from the group consisting of a chemical bond, an avidin-biotin interaction, a hydrophobic interaction, an electrostatic interaction, and an affinity interaction to obtain the composite particle for an immunochromatography.

16. An immunochromatography using the composite particle for an immunochromatography according to claim 2.

17. The immunochromatography according to claim 16, comprising:
a mixing step of mixing a specimen that contains a test substance with a modified composite particle that is the composite particle for an immunochromatography, which is modified with a first binding substance binding to the test substance, to obtain a complex of the test substance in the specimen and the modified composite particle;
a collection step of collecting, using magnetism, the complex in the specimen obtained after the mixing step;
a spreading step of spreading the complex collected in the collection step on an insoluble carrier having a reaction site at which a second binding substance binding to the test substance is immobilized; and
a trapping step of trapping the complex at the reaction site of the insoluble carrier.

18. The immunochromatography according to claim 17, further comprising:
a silver amplification step of silver-amplifying the trapped complex subsequent to the trapping step.

19. The composite particle for an immunochromatography according to claim 3,
wherein an average number of the gold particles bound to one of the magnetic particles via the organic substance is less than 10.0.

20. The composite particle for an immunochromatography according to claim 19,
wherein an average number of the gold particles bound to one of the magnetic particles via the organic substance is 8.0 or less.
